(19) United States
(12) Patent Application Publication
(10) Pub. No.: US 2004/0147775 A1
(43) Pub. Date: Jul. 29, 2004

(54) Process for the synthesis of 3.3A.6.6A-tetrahydro-2H-cyclopentan[b]furan-2-one

(76) Inventors: Kevin Edward Henegar, Ann Arbor, MI (US); Mateusz Cebula, Portage, MI (US)

Correspondence Address: PHARMACIA & UPJOHN, 301 HENRIETTA ST, 0228-32-LAW, KALAMAZOO, MI, 49007, US

(21) Appl. No.: 10/735,125

(22) Filed: Dec. 12, 2003

Related U.S. Application Data
(60) Provisional application No. 60/435,991, filed on Dec. 23, 2002.

(51) Int. Cl. C07C 235/30

(52) U.S. Cl. 560/250

(57) ABSTRACT

The present invention relates to a process for the synthesis of 3,3a,6,6a-tetrahydo-2H-cyclopentan[b]furan-2-one.

### CROSS REFERENCE TO RELATED APPLICATIONS

This application claims the benefit of U.S. provisional application Serial No. 60/435,991 filed on 23 Dec. 2002, under 35 USC 119(e)(i), which is incorporated herein by reference in its entirety.

### FIELD OF THE INVENTION

This invention relates to a process for the synthesis of 3,3a,6,6a-tetrahydo-2H-cyclopentan[b] furan-2-one, a molecule that is useful as an intermediate in the synthesis of prostaglandins.

### BACKGROUND

The prostaglandins are an important series of molecules that have a wide variety of uses. There are many known syntheses of prostaglandins and 3,3a,6,6a-tetrahydo-2H-cyclopentan[b]furan-2-one is a known intermediate in several such syntheses. 3,3a,6,6a-Tetrahydo-2H-cyclopentan[b]furan-2-one (lactone) has been prepared by a number of means. Racemic material can be prepared by the reaction of dichloroketene with cyclopentadiene followed by dechlorination with zinc followed by a Bayer-Williger oxidation (Grieco, P. A., J. Org. Chem. 1972, 37, 2363-4). This process suffers from the problem of formation of black tars in the dichloroketene step and requires a resolution as has been described (Corey, E. J.; Snider, B. B., J. Org. Chem. 1974, 39, p 256-8; Covington, E. W. et al., Tetrahedron Lett. 1983, 3125-3128; Carnell, A. J., et al., J. Chem. Soc., Chem. Commun. 1990, 20, 1438-9; Bertolasi, V., et al., Tetrahedron: Asymmetry (2001), 12(10), 1479-1483).

Another method for the preparation of 3,3a,6,6a-tetrahydo-2H-cyclopentan[b]furan-2-one in enantiomerically enriched form involves an asymmetric hydroboration of cyclopentadieneacetic acid (Partridge, J. J., et al., J. Am. Chem. Soc. 1973, 95, 7171-2; Partridge, J. J., et al., Org. Syn. Coll. Vol. VII, 339-345). This procedure has the advantage of avoiding a resolution but has a number of operational problems that limit its potential as a large-scale production process.

A third method is Claisien rearrangement of a 3-acyloxy-5-hydroxycyclopentene. Orthoester Claisien rearrangements on the 3S,5R monoacetate have been described (Laumen, K., et al., J. Chem. Soc. Chem. Comm. 1986, 1298-1299; Laumen, K., et al., Tetrahedron Lett. 1984, 25, 5875-5878; Nara, M., et al., Tetrahedron, 1980, 36, 3161-3170; Takano, S., et al., J. Chem. Soc., Chem. Commun. 1976, 6, 189-190) but require the use of high temperature (160° C.) which is difficult to achieve on a production scale.

Accordingly, there is a need for a simple economical process for the production of 3,3a,6,6a-tetrahydo-2H-cyclopentan[b]furan-2-one which is economical, provides enantiomerically enriched product and is suitable for large-scale production.

### SUMMARY OF THE INVENTION

The present invention provides a process for the production of 3,3a,6,6a-tetrahydo-2H-cyclopentan[b]furan-2-one (Formula IV) comprising the steps:

a) Reacting a 3-acyloxy-5-hydroxycyclopentene of Formula I

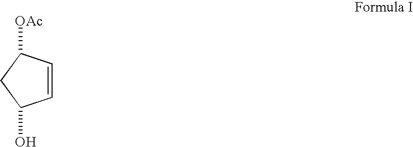

with an amide acetal of Formula IIa or a ketene aminoacetal of Formula IIb

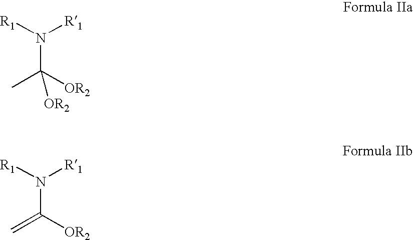

wherein;

R₁ and R′₁, are C₁ to C₄ alkyl or

R₁ and R′₁, taken together form a ring of 3 to 7 members;

R₂ is C₁ to C₄ alkyl;

Ac is C₁ to C₄ alkanoyl;

at 90-120° C. in a solvent while maintaining an alcohol (R₂OH) concentration of less than 3% by volume to give an acylhydroxycyclopenteneacetamide of Formula III;

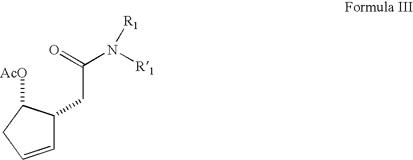

b) Adding an alkali or alkali earth hydroxide, carbonate, or quaternary ammonium hydroxide solution to give a homogeneous or biphasic mixture; and

c) Adding a strong acid of pKₐ<2 to give the lactone of Formula IV.

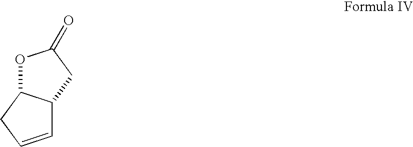

### DETAILED DESCRIPTION OF THE INVENTION

The present invention provides a process for the production of 3,3a,6,6a-tetrahydo-2H-cyclopentan[b]furan-2-one comprising the steps:

a) Reacting a 3S,5R 3-acyloxy-5-hydroxycyclopentene of Formula I

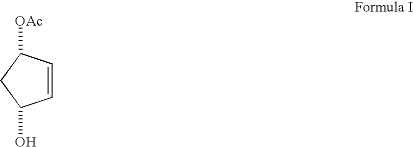

with an amide acetal of Formula IIa or a ketene aminoacetal of Formula IIb

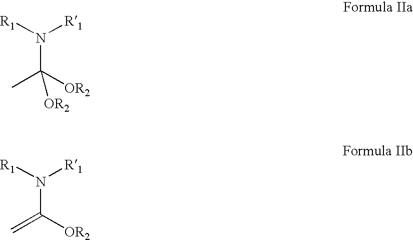

wherein;

R₁ and R′₁, are C₁ to C₄ alkyl or

R₁ and R′₁, taken together form a ring of 3 to 7 members;

R₂ is C₁ to C₄ alkyl;

Ac is C₁ to C₄ alkanoyl;

at 90-140° C. in a suitable solvent of boiling point >90° C. while maintaining an alcohol (R₂OH) concentration of less than 3% by volume to give a acylhydroxycyclopenteneacetamide of Formula III;

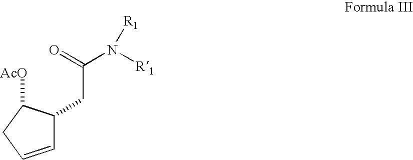

b) Adding an alkali or alkali earth hydroxide, carbonate, or quaternary ammonium hydroxide solution to give a homogeneous or biphasic mixture; and

c) Adding a strong acid of pKₐ<2 to give the lactone of Formula IV.

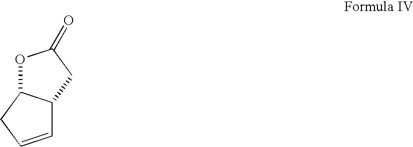

Use of an amideacetal of Formula IIa or a ketene amino acetal of Formula IIb to affect the Claisien rearrangement in Step a leads to the surprising result that the rearrangement under these conditions requires a temperature of only 90-120° C., a temperature readily achieved in normal production equipment.

A further unexpected result is that maintaining an alcohol (R₂OH) concentration in the reaction mixture below 3% minimizes racemization. As shown in Chart 1, concentrations of alcohol greater than 3-5% lead to extensive racemization of the starting acyloxyhydroxycyclopentene of Formula I. The chart depicts the racemization that occurs after 1 hour reaction time when conducting Step a with dimethylacetamide dimethylacetal (DMA) in toluene, a bath temperature of 120° C. and varying amounts of methanol.

[image]

As Chart 1 shows, it is important to maintain an alcohol concentration in the reaction mixture below 3%, and preferably below 2%. This can be accomplished by ensuring a low concentration of alcohol in the starting materials, distillation of the product alcohol from the reaction mixture, optionally, adding the acetal or ketene acetal in small portions, and, optionally, purging the head space of the reactor with an inert gas such as nitrogen, argon and the like.

In Step a, other suitable solvents besides toluene include xylene, mesitylene, anisole, chlorobenzene, bromobenzene, o-dichlorobenzene, ethylbenzene, indan, tetralin, decalin, heptane, octane, isooctane, and higher alkanes, methylcyclohexane, dimethylcyclohexanes, ethylcyclohexane bromobutane or other higher boiling haloalkanes ethyleneglycol dimethyl ether, ethyleneglycol diethyl ether, higher ethylene glycol ethers, diethyleneglycol ethers, propyl ether, butyl ether and similar higher boiling ethers, dimethyltetrahydrofuran, 1,4-dioxane, 2,2-dimethyl-1,3-dioxolane, acetaldehyde diethylacetal and similar higher boiling acetals, triethylamine and higher boiling tertiary amines, dimethylaniline, pyridine, lutidines, collidines, n-methylpiperidine, quinoline, quinaldine, lepidine, isoquinoline propionitrile, benzonitrile, and similar higher boiling nitriles and the like having a boiling point of >90° C. at normal pressure. Non-limiting examples of amide acetals include dimethylacetamide dimethylacetal, dimethylacetamide diethylacetal, diethylacetamide dimethylacetal and N-1,1-dimethoxyethylpyrrolidine. Non-limiting examples of ketene aminoacetals include 1-methoxy-1-dimethylaminoethylene, and 1-methoxy-1-diethylaminoethylene, 1-ethoxy-1-diethylaminoethylene and 1-methoxy-1-pyrrolidinylethylene. The amides of Formula III may be isolated and purified by chromatography or, usually, they may be used in crude form for Step b.

In Step b, suitable bases include sodium hydroxide, potassium hydroxide, lithium carbonate, cesium carbonate, tetrabutylammonium hydroxide and the like in aqueous solution. Under biphasic conditions, a phase transfer catalyst such as benzyltridecylammonium hydroxide and the like may optionally be used. The intermediate product of amide hydrolysis is normally not isolated, but is converted directly to the lactone of Formula IV by acidification of the alkaline hydrolysis mixture.

In the final Step c, suitable acids of pKₐ of <2 for acidification include hydrochloric, sulfuric, hydrobromic, phosphoric, fluoboric, perchloric, toluene sulfonic, methane sulfonic, trifluroacetic, and the like in aqueous solution. The title compound is isolated with conventional techniques such as extraction, chromatography and crystallization.

Without further elaboration, one skilled in the art can, using the preceding description, practice the present invention to its fullest extent. The following detailed examples describe how to prepare the various compounds and perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### EXAMPLE 1

### 1S,5R-2-oxabicyclo[3.3.0]oct-6-en-3-one.

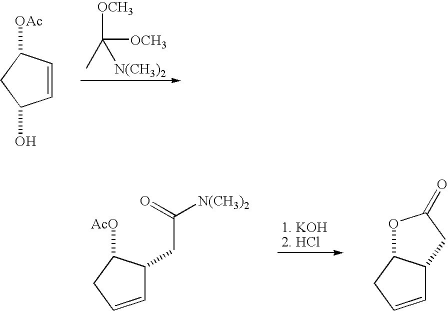

Step a: (1R-cis) 5-(acetyloxy)-N,N-dimethyl-2-cyclopentene-1-acetamide.

A mixture of 3S,5R 3-acetoxy-5-hydroxycyclopentene (Aldrich Chemical Co., 17.55 g, 123 mmole, 98.8% ee), dimethylacetamide dimethylacetal (Aldrich Chemical Co, 2 4.57 g, 182 mmole), and 351 mL of toluene is heated in an oil bath maintained at 130° with distillation of methanol through a short path condenser. After the initial distillation of methanol from the mixture a slow nitrogen sweep was begun and additional toluene was added as needed to maintain the original volume. Heating was continued for 6 to 8 hours and the toluene was removed by distillation to give a crude product as a dark red-brown oil. [(5-(acetyloxy)-N,N-dimethyl-2-cyclopentene-1-acetamide has been described; Ema, T., et al., J. Org. Chem., 1996, 61, 8610] ¹H NMR (CDCl₃) [symbol] 2.01 (s, 3H), 2.31-2.37 (m, 2H), 2.55 (dd, 1H, J=16.0, 7.0 2.75 (dd, 1H, J=16, 6.6 Hz), 2.96 (s, 3H), 3.02 (s, 3H), 3.38, (m, 1H), 5.46 (m, 1H)5,73 (m, 2H).

¹³C NMR (CDCl₃) [symbol] 21.42, 32.30, 35.77, 37.59, 39.57, 44.74, 75.42, 128.46, 133.41, 170.83, 172.03

Step b: (1R-cis) 5-hydroxy-2-cyclopentene-1-acetic acid, potassium salt.

The crude amide from Step a was dissolved in 50 mL of MTBE. A solution of potassium hydroxide (16.2 g, 246 mmole) in 160 mL of water was added and the mixture was heated in a 65° C. bath for 1 hour with stirring. The mixture was cooled and the phases separated. The aqueous phase was washed with MTBE (50 mL) to give an aqueous solution of the title compound.

Step c: 1S,5R-2-oxabicyclo[3.3.0]oct-6-en-3-one.

The alkaline solution of Step b was acidified to a pH of 1.0 to 1.5 with concentrated hydrochloric acid and stirred for 1.0 hour. The mixture was extracted with methylene chloride (3×50 mL) and the organic extract concentrated to 50-70 mL and filtered through silica gel (10 g, 230-400 mesh). The silica gel was washed with additional methylene chloride (75 mL). The combined filtrates were concentrated at 30° C. (bath) under reduced pressure (100 mm) to yield the lactone which crystallizes on standing. ¹H NMR (CDCl₃) □2.17 (d, 1H, J=18 Hz), 2.39-2.55 (m, 4H), 3.26 (m, 1H), 4.87 (t, 1H, J=5.6 Hz), 5.33 (m, 1H), 5,52 (m, 1H).

¹³C NMR (CDCl₃) [symbol] 33.71, 39.85, 45.83, 83.41, 129.9, 131.7, 177.1.

### EXAMPLE 2

### Slow Addition of Dimethylacetamide Dimethylacetal

Step a: (1R-cis) 5-(acetyloxy)-N,N-dimethyl-2-cyclopentene-1-acetamide.

30.0 g of 3S,5R 3-acetoxy-5-hydroxycyclopentene was dissolved in 240 mL of toluene and the solution was filtered through magnesol to remove a small amount of insoluble material. The filtered solution was heated to 100° C. (internal temperature) and a solution of 64.6 mL of dimethylacetamide dimethyl acetal, (28% (v/v) methanol) in 64.5 mL of toluene was added in portions over 6 hours while maintaining a slow distillation rate. The reaction mixture was heated for an additional 4 hours after completion of the addition. The mixture was then concentrated under vacuum to yield the product as a dark oil.

Step b: (1R-cis) 5-hydroxy-2-cyclopentene-1-acetic acid, potassium salt.

The oil was dissolved in 85 mL of MTBE and 27.7 g of potassium hydroxide and 109 mL of water were added. The two-phase mixture was heated under reflux for 1 hour. The phases were separated and the aqueous phase was extracted with 85 mL of MTBE to give an aqueous solution of the title compound.

Step c: 1S,5R-2-oxabicyclo[3.3.0]oct-6-en-3-one.

50 mL of concd. hydrochloric acid was added to the aqueous phase of Step b (final pH 1.0) and the mixture was stirred for 1 hour at room temperature. The mixture was extracted with methylene chloride (3×120 mL). The combined methylene chloride solutions were filtered through silica (17 g, 230-400 mesh). The filtrate was evaporated to yield 23.91 g (91.2% overall yield).

1. A process for preparing a 5-(acyloxy)-N,N-dialkyl-2-cyclopentene-1-acetamide of Formula III comprising:
Reacting a 3-acyloxy-5-hydroxycyclopentene of Formula I 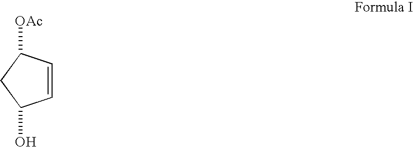
with an amide acetal of Formula IIa or a ketene aminoacetal of Formula IIb 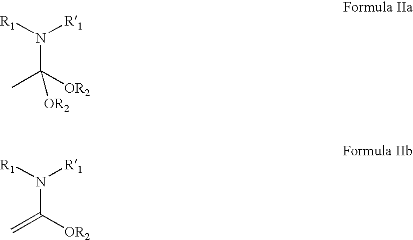
wherein;
R₁ and R′₁, are C₁ to C₄ alkyl or
R₁ and R′₁, taken together form a ring of 3 to 7 members;
R₂ is C₁ to C₄ alkyl;
Ac is C₁ to C₄ alkanoyl;
at 90-140° C. in a suitable solvent of boiling point >90° C. while maintaining an alcohol R₂OH concentration of less than 3% by volume to give an acylhydroxycyclopenteneacetamide of Formula III;

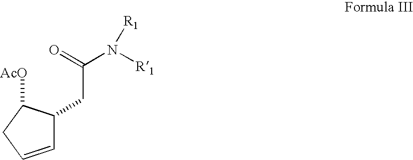

2. A process according to claim 1 for preparing (4R, 5S)-3,3a,6,6a-tetrahydo-2H-cyclopentan[b]furan-2-one further comprising the steps of:
Adding an alkali or alkali earth hydroxide, carbonate, bicarbonate, or quaternary ammonium hydroxide solution to give a homogeneous or biphasic mixture; and
Adding a strong acid of pKₐ<2 to give the title lactone of Formula IV.

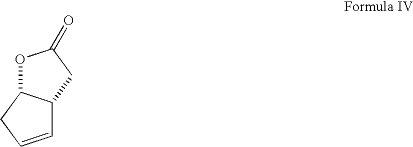

3. A product prepared by a process comprising:
Reacting a 3-acyloxy-5-hydroxycyclopentene of Formula I 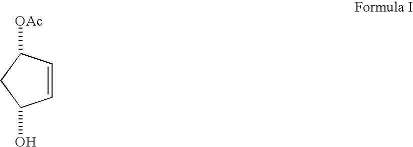
with an amide acetal of Formula IIa or a ketene aminoacetal of Formula IIb 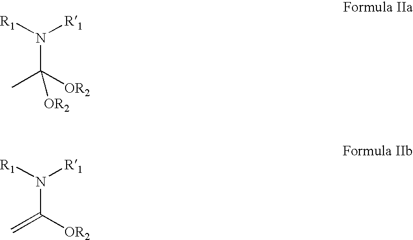
wherein;
R₁ and R′₁, are C₁ to C₄ alkyl or
R₁ and R′₁, taken together form a ring of 3 to 7 members;
R₂ is C₁ to C₄ alkyl;
Ac is C₁ to C₄ alkanoyl;
at 90-140° C. in a suitable solvent of boiling point >90° C. while maintaining an alcohol R₁OH concentration of less than 3% by volume to give an acylhydroxycyclopenteneacetamide of Formula III;

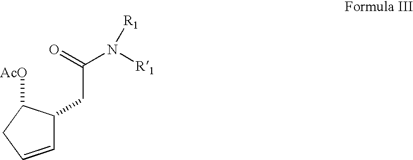

4. A product prepared by a process according to claim 3 further comprising the steps of:
Adding an alkali or alkali earth hydroxide, carbonate, bicarbonate, or quaternary ammonium hydroxide solution to give a homogeneous or biphasic mixture; and
Adding a strong acid of pKₐ<2 to give a lactone of Formula IV. [chemical structure]
